(19) United States
(12) Patent Application Publication
(10) Pub. No.: US 2025/0221718 A1
(43) Pub. Date: Jul. 10, 2025

(54) Powered Surgical Drill Having A Depth Measurement Extension

(71) Applicant: Stryker Corporation, Kalamazoo, MI (US)

(72) Inventors: Trevor Jonathan Lambert, Portage, MI (US); Gerardus Franciscus Knijnenburg, Fermoy, Co. Cork (IE)

(73) Assignee: Stryker Corporation, Kalamazoo, MI (US)

(21) Appl. No.: 18/701,872

(22) PCT Filed: Oct. 19, 2022
(86) PCT No.: PCT/US2022/047132

§ 371 Date: Apr. 16, 2024

Related U.S. Application Data
(60) Provisional application No. 63/257,248, filed on Oct. 19, 2021.

(51) Int. Cl. A61B 17/16; A61B 90/00

(52) U.S. Cl. CPC A61B 17/1615; A61B 17/1628; A61B 17/1633; A61B 90/06; A61B 2090/062

(57) ABSTRACT

A surgical handpiece system. The surgical handpiece system includes a drill bit having a shank portion and a cutting portion. The surgical handpiece system also includes a surgical handpiece assembly. The surgical handpiece assembly includes a motor and a drive element configured to rotate the drill bit. A depth measurement member is moveable along an axis of the drill bit. A displacement sensor generates displacement signals responsive to movement of the depth measurement member. A depth measurement extension is configured to be coupled to the depth measurement member. The depth measurement extension has an elongated body for receiving the drill bit. The elongated body is sized to accommodate the shank and cutting portions of the drill bit. The elongated body is also sized to prevent passage of the drill bit entirely through the elongated body.

### CROSS REFERENCE TO RELATED APPLICATIONS

This application claims priority to and all the advantages of U.S. Provisional Patent Application No. 63/257,248, filed on Oct. 19, 2021, the entire contents of which are hereby incorporated by reference.

### BACKGROUND

Conventional medical and surgical procedures routinely involve the use of surgical tools and instruments which allow surgeons to approach and manipulate surgical sites. By way of non-limiting example, rotary instruments such as handheld drills are commonly utilized in connection with orthopedic procedures to address various musculoskeletal conditions, such as trauma, sports injuries, degenerative diseases, joint reconstruction, and the like. In procedures where handheld drills or similar surgical instruments are employed, rotational torque selectively generated by an actuator (e.g., an electric motor) is used to rotate a releasably-attachable drill bit or other surgical attachments at different speeds.

A surgical handpiece assembly drills bores in the tissue against which the drill bit is applied. One type of surgical procedure in which it is necessary to drill a bore is a trauma procedure to repair a broken bone. In this type of procedure, an elongated rod, sometimes called a nail, is used to hold the fractured sections of the bone together. To hold the nail in place, one or more bores are driven into the bone. These bores are positioned to align with complementary holes formed in the nail. A screw is inserted in each aligned bore and nail hole. The screws hold the nail in the proper position relative to the bone.

In another type of procedure, an implant known as a plate is secured to the outer surfaces of the fractured sections of a bone to hold the sections together. Screws hold the plate to the separate sections of bone. To fit a screw that holds a plate to bone it is necessary to first drill a bore to receive the screw.

As part of a procedure used to drill a screw-receiving bore in a bone, it is desirable to know the end-to-end depth of the bore. This information allows the surgeon to select size of screw that is fitted in the bore hole. If the screw is too short, the screw may not securely hold the nail into which the screw is inserted in place. If the screw is too long, the screw can extend an excessive distance out beyond the bone. If the screw extends an excessive distance beyond the bone, the exposed end of the screw can rub against the surrounding tissue. If this event occurs, the tissue against which the screw rubs is affected.

While surgical drills are routinely utilized to assist in the performance of a variety of different types of medical and/or surgical procedures, there is a need in the art to continuously improve such surgical drills.

### SUMMARY

The present disclosure provides a surgical handpiece system. The surgical handpiece system comprises a drill bit having a shank portion and a cutting portion. The surgical handpiece system further comprises a surgical handpiece assembly. The surgical handpiece assembly comprises a handpiece housing and a motor disposed within the handpiece housing. The motor is configured to generate a torque. The surgical handpiece assembly further comprises a drive element disposed within the handpiece housing and configured to be coupled to the drill bit. The drive element is configured to receive the torque from the motor and rotate the drill bit in response to the torque when the drill bit is coupled to the drive element. The surgical handpiece system further comprises a depth measurement member being moveable relative to the handpiece housing along an axis that is parallel to an axis of the drill bit. The surgical handpiece system further comprises a displacement sensor for generating displacement signals responsive to movement of the depth measurement member relative to the handpiece housing. The surgical handpiece system further comprises a depth measurement extension configured to be coupled to the depth measurement member. The depth measurement extension comprises an elongated body having an inner surface defining a bore extending between proximal and distal ends for receiving the drill bit. The elongated body comprises a proximal portion sized to accommodate passage of the shank portion of the drill bit through the bore. The proximal portion is sized to prevent passage of the cutting portion of the drill bit through the bore. The elongated body comprises a distal portion sized to accommodate passage of the shank portion and the cutting portion of the drill bit through the bore.

The present disclosure also provides a surgical handpiece system. The surgical handpiece system comprises a first drill bit having a first shank portion and a first cutting portion. The first cutting portion has a first outer diameter. The surgical handpiece system comprises a second drill bit having a second shank portion and a second cutting portion. The second cutting portion has a second outer diameter greater than the first outer diameter. The surgical handpiece system comprises a surgical handpiece assembly. The surgical handpiece assembly comprises a handpiece housing and a motor disposed within the handpiece housing. The motor is configured to generate a torque. The surgical handpiece assembly comprises a drive element disposed within the handpiece housing. The drive element is configured to be coupled separately to each of the first and second drill bits. The drive element is configured to receive the torque from the motor and rotate one of the first and second drill bits in response to the torque when one of the first and second drill bits is coupled to the drive element. The surgical handpiece system comprises a depth cannula being moveable relative to the handpiece housing. The depth cannula defines a lumen extending along a length of the depth cannula. The depth cannula is sized to separately receive at least the first and second shank portions of the first and second drill bits. The depth cannula has a first inner diameter greater than the first outer diameter of the first cutting portion of the first drill bit. The first inner diameter is less than the second outer diameter of the second cutting portion of the second drill bit.

The surgical handpiece system comprises a displacement sensor for generating displacement signals responsive to movement of the depth cannula relative to the handpiece housing. The surgical handpiece system comprises a depth measurement extension configured to be coupled to the depth cannula. The depth measurement extension comprises an elongated body. The elongated body has a distal portion having a second inner diameter greater than the first and second outer diameters of the first and second cutting portions of the first and second drill bits.

The present disclosure also provides a depth measurement extension for coupling to a depth measurement member of a surgical drilling system that includes a drill bit having a shank portion and a cutting portion. The depth measurement extension comprises an elongated body having an inner surface that defines a bore extending between proximal and distal ends for receiving the drill bit and the depth measurement member. The elongated body comprises a proximal portion having a first internal cross-sectional area for accommodating the shank portion of the drill bit. The elongated body comprises a distal portion having a second internal cross-sectional area for accommodating the cutting portion of the drill bit. The second internal cross-sectional area is greater than the first internal cross-sectional area. The elongated body comprises a coupling portion disposed proximal of the proximal portion. The coupling portion has a third internal cross-sectional area for accommodating the depth measurement member. The third internal cross-sectional area is greater than the first internal cross-sectional area.

### BRIEF DESCRIPTION OF THE DRAWINGS

FIG. 1 is perspective view of a surgical handpiece system comprising a surgical handpiece assembly and a measurement module, the surgical handpiece assembly shown having a drill bit according to one configuration.

FIG. 2 is a partially-exploded perspective view of the surgical handpiece system of FIG. 1, with the surgical handpiece system shown having a measurement module, a drive cannula, a depth measurement extension, and a release assembly spaced from a handpiece housing assembly, and with the end effector assembly removed from the surgical handpiece assembly.

FIG. 3 is a partially-exploded perspective view of portions of the surgical instrument of FIGS. 1-2, shown with the drive assembly and the release mechanism spaced from a phantom outline of the handpiece body to depict an actuator assembly.

FIG. 4 is a partial isometric sectional view taken along line 4-4 in FIG. 1 illustrating the arrangement of the depth measurement extension attached to the measurement module.

FIG. 5A is a sectional view taken longitudinally through the surgical instrument of FIGS. 1-4 illustrating the arrangement of the depth measurement extension attached to the measurement module with the depth measurement extension in a first position.

FIG. 5B is a sectional view taken longitudinally through the surgical instrument of FIGS. 1-4 illustrating the arrangement of the depth measurement extension attached to the measurement module with the depth measurement extension in a second position.

FIG. 6 is a sectional view of the depth measurement extension and the drill bit of the surgical handpiece system.

FIG. 7A is a front elevation view of the depth measurement extension.

FIG. 7B is a sectional view taken along line 7B-7B in FIG. 7A illustrating the depth measurement extension.

FIG. 8A is another front elevation view of the depth measurement extension.

FIG. 8B is a sectional view taken along line 8B-8B in FIG. 8A illustrating

the depth measurement extension.

### DETAILED DESCRIPTION

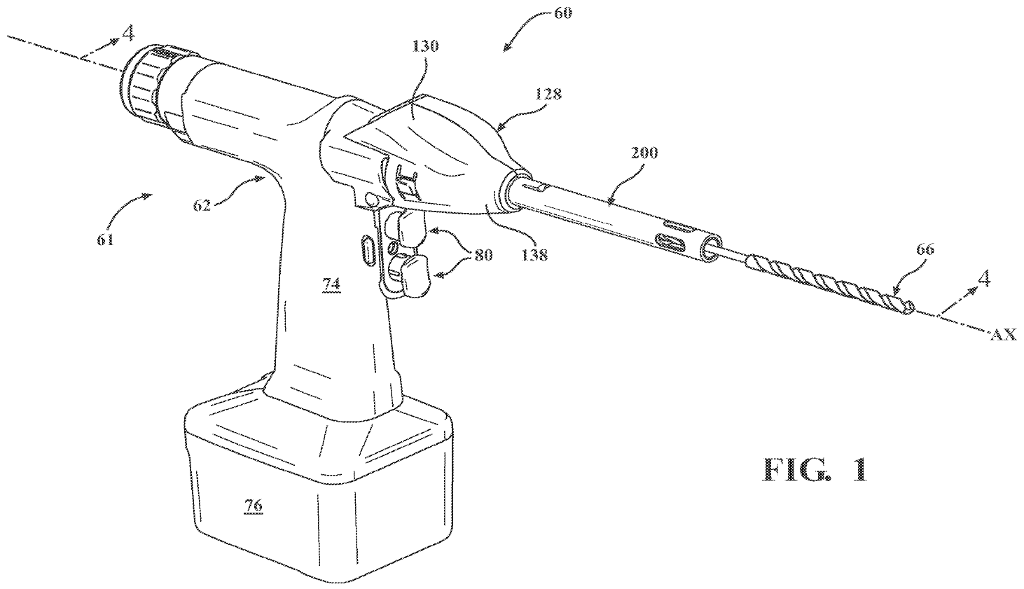

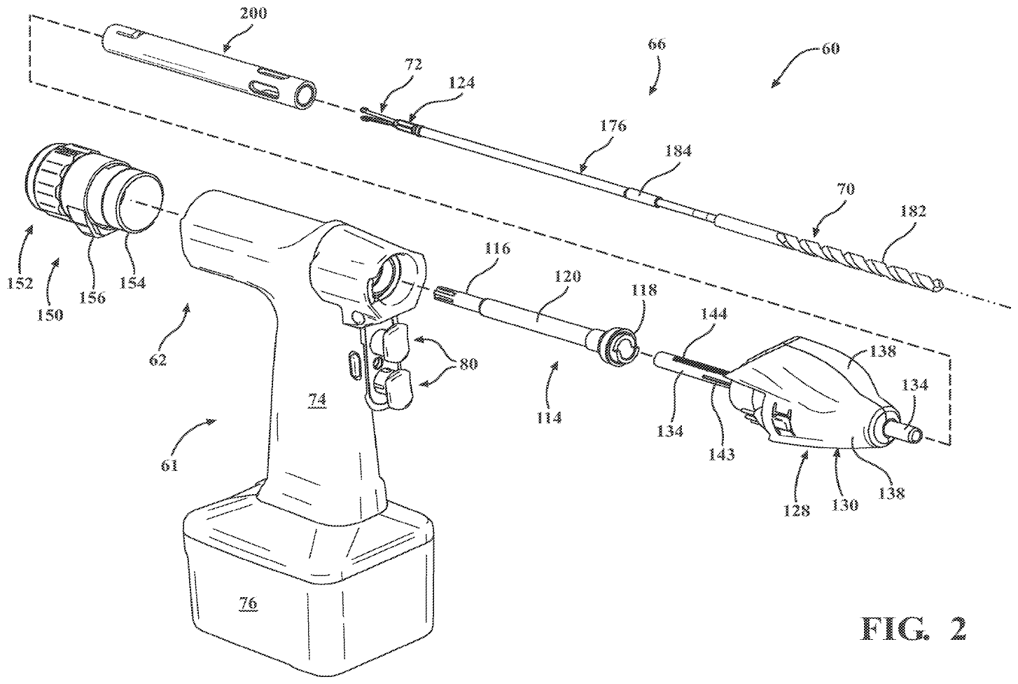

With reference to the drawings, where like numerals are used to designate like structure throughout the several views, a surgical system, or surgical drill system, is shown at 60 in FIGS. 1-2 for performing an operational function associated with medical and/or surgical procedures. In some configurations, the surgical drill system 60 may also be referred to as a surgical handpiece system. In the representative configuration illustrated herein, the surgical handpiece system 60 is employed to facilitate penetrating a workpiece, such as tissue or a bone of a patient. To this end, the illustrated configuration of the surgical handpiece system 60 comprises a surgical drill 61 that comprises a handpiece 62, alternatively referred to as a handheld surgical instrument, and a drill bit 66. As is best depicted in FIG. 2, the drill bit 66 extends generally longitudinally along an axis AX between a cutting tip portion, generally indicated at 70, and an insertion portion, generally indicated at 72. As is described in greater detail below, the cutting portion 70, sometimes referred to as a cutting tip portion, is configured to engage tissue, and the insertion portion 72 is configured to facilitate releasable attachment of the drill bit 66 to the surgical handpiece assembly 62. Various configurations of the insertion portion 72 are contemplated to enable coupling of the drill bit 66 to the handpiece 62, such as various grooves, slots, and other geometries. One exemplary configuration of an insertion portion can be found in U.S. Pat. No. 10,159,495, which is hereby incorporated by reference in its entirety. It is contemplated that there may be other configurations to facilitate attachment of the drill bit 66 to the handpiece 62.

As also shown in FIG. 2, the drill bit 66 extends along the axis AX from a proximal end to a distal end. The cutting portion 70 may define flutes 182 which may be helically disposed about the axis AX and extend proximally from a distal end of the drill bit 66 to promote workpiece, such as tissue, penetration (see FIG. 2). The drill bit 66 comprises a shank portion, generally indicated at 176, which extends along the axis AX between the insertion portion 72 and the cutting portion 70. The shank portion 176 may vary in thickness along its length. In the configuration illustrated in FIG. 2, the shank portion 176 of the drill bit 66 may also include a bearing region 184 coupled to the shank portion 176. The bearing region 184 is sized so as to be received within and rotate relative to a depth cannula 134 of a measurement module 128 that may be coupled to the handpiece (discussed in greater detail further below). Here, the bearing region 184 may define a “stepped” outer region of the shank portion 176 that affords rotational support along the length of the drill bit 66, and may have a larger diameter than adjacent distal and proximal regions of the shank portion 176 in the illustrated configuration. However, it will be appreciated that the bearing region 184 of the shank portion 176 of the drill bit 66 could be configured in other ways without departing from the scope of the present disclosure.

Referring now to FIGS. 1-4, in the representative configuration illustrated herein, the handpiece 62 is realized as a handheld drill with a pistol-grip shaped handpiece body 74 which releasably attaches to a battery 76 (battery attachment not shown in detail). However, it is contemplated that the handpiece body 74 can have any suitable shape with or without a pistol grip. While the illustrated handpiece 62 employs a battery 76 which is releasably attachable to the handpiece body 74 to provide power to the handpiece 62 utilized to rotate the drill bit 66, it will be appreciated that the handpiece 62 may be configured in other ways, such as with an internal (e.g., non-removable) battery, or with a tethered connection to an external console, power supply, and the like. Other configurations are contemplated.

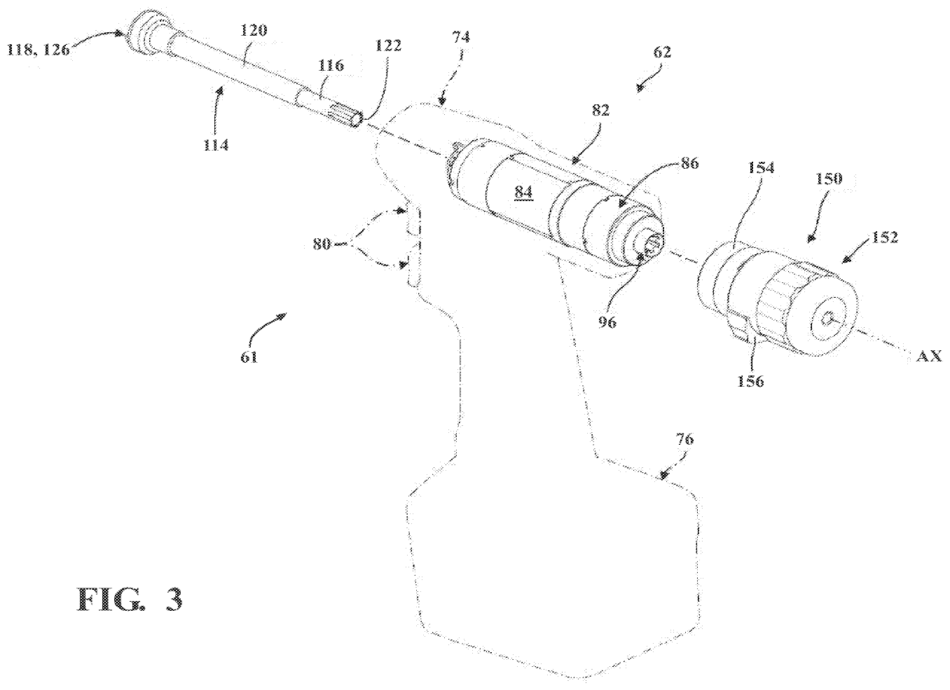

In the illustrated configuration, the battery 76 or other power source provides power to a controller 78, which is disposed in communication with an input control 80 and an actuator assembly 82 (see also FIG. 3). The input control 80 and the actuator assembly 82 are each supported by the handpiece body 74. The controller 78 is generally configured to facilitate operation of the actuator assembly 82 in response to actuation of the input control 80. The input control 80 has a trigger-style configuration in the illustrated configuration, is responsive to actuation by a user (e.g., a surgeon), and communicates with the controller 78, such as via electrical signals produced by magnets and Hall effect sensors. Thus, when the operator actuates the input control 80 to operate the handpiece 62, the controller 78 directs power from the battery 76 to the actuator assembly 82 which, in turn, generates rotational torque employed to rotate the drill bit 66, as described in greater detail below. The handpiece body 74, the battery 76, the controller 78, and the input control 80 could each be configured in a number of different ways to facilitate generating rotational torque without departing from the scope of the present disclosure.

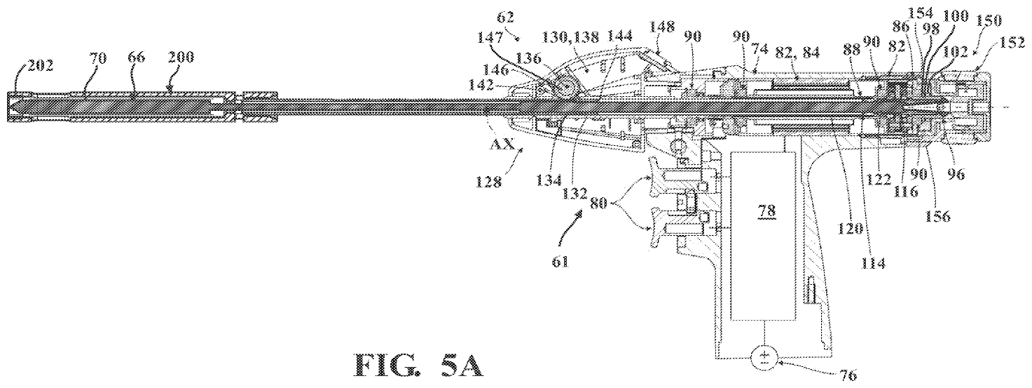

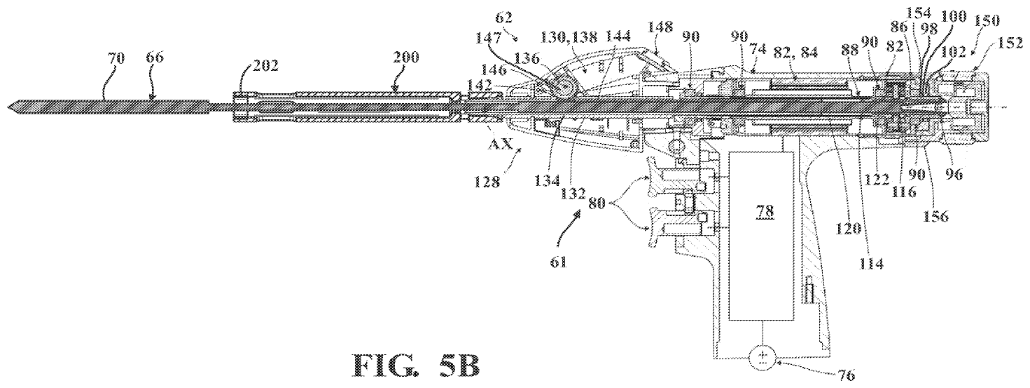

Referring As also shown in FIG. 3, the actuator assembly 82 may comprise an electric motor 84 and a gearset 86 which are each supported within the handpiece body 74. The motor 84 is configured to selectively generate rotational torque in response to commands, signals, and the like received from the controller 78. As is best shown in FIG. 5, the motor 84 comprises a rotor cannula 88 supported for rotation about the axis AX by a pair of bearings 90. A drive gear arranged adjacent to the gearset 86 is coupled to and rotates concurrently with the rotor cannula 88, and is employed to transmit rotational torque to the gearset 86. To this end, in the illustrated configuration, the gearset 86 is realized as two-stage compound planetary arrangement and generally comprises a ring gear housing 94 which, among other things, rotationally supports an output hub 96 via a bearing 90, as well as one or more retaining clips 98, washers 100, and/or seals 102. However, other configurations of the gearset 86 are contemplated.

Further details of one configuration of a gearset 86 are described, for example, in U.S. patent application Ser. No. 15/887,507, filed on Feb. 2, 2018 and entitled “Drill Bit for Handheld Surgical Instrument, the contents of which are herein incorporated by reference in their entirety, and describe wherein the rotation of the drive gear via actuation of the motor 84 effects concurrent rotation of the output hub 96, and wherein the output hub 96 rotates concurrently with the drill bit 66. The actuator assembly 82 could be configured in other ways without departing from the scope of the present disclosure. By way of non-limiting example, while the illustrated actuator assembly 82 employs a compound planetary arrangement to adjust rotational speed and torque between the drive gear of the motor 84 and the output hub 96, other types of gearsets 86 could be utilized in some configurations. Moreover, while the illustrated actuator assembly 82 employs an electrically-powered brushless DC motor to generate rotational torque, other types of prime movers could be utilized. Other configurations are contemplated.

As noted above, rotational torque generated by the motor 84 effects rotation of the output hub 96 which, in turn, rotates concurrently with the coupled drill bit 66. To this end, and as is best shown in FIGS. 2-5, the handpiece 62 further comprises a drive assembly 114 which generally extends through the various cannulated components of the actuator assembly 82 into splined engagement with the output hub 96 of the gearset 86. The drive assembly 114 is configured to facilitate releasable attachment between the drill bit 66 and the handpiece 62. The drive assembly 114 generally comprises a drive element 116 such as a drive cannula, a driving head 118, and a driving body 120 which extends between, and rotates concurrently with, the drive element 116 and the driving head 118. The drive assembly 114 is supported for rotation about the axis AX within the handpiece body 74 via splined engagement with the output hub 96 adjacent the drive element 116, and via an arrangement of bearings, washers, and seals adjacent the driving head 118. It is contemplated that the drill bit 66 may be configured to attach to the handpiece 62 to receive torque in a manner different from that described above.

Further details of the drive assembly 114 are also described, for example, in U.S. patent application Ser. No. 15/887,507, the contents of which are also herein incorporated by reference in their entirety. In the illustrated configuration, the driving head 118 of the drive assembly 114 comprises a coupling, generally indicated at 126, which is provided to facilitate transmitting rotational torque when the handpiece 62 is utilized in connection with other applications besides rotating the drill bit 66 of the present disclosure. More specifically, the illustrated drive assembly 114 is configured such that the handpiece 62 can rotate, drive, or otherwise actuate a number of different types of surgical instruments, tools, modules, end effectors, and the like, which can be configured to engage and rotate concurrently with either the bore 122 of the drive element 116, or the coupling 126 of the driving head 118. It will be appreciated that this configuration allows the same handpiece 62 to be utilized in a broad number of medical and/or surgical procedures. However, it is contemplated that the drive assembly 114 could be configured differently in some configurations, such as to omit a driving head 118 with a coupling 126 in configurations where the handpiece 62 configured for dedicated use with the drill bit 66 of the present disclosure.

Referring back to FIGS. 1-3, the illustrated configuration of the handpiece 62 further comprises a release mechanism, or coupling mechanism, generally indicated at 150, configured to facilitate removal of the drill bit 66. The coupling mechanism 150 generally comprises a release subassembly 152, a keeper body 154, and a housing adapter 156. The keeper body 154 and the housing adapter 156 are respectively configured to secure the release subassembly 152 to the actuator assembly 82 and the handpiece body 74, and could be realized with a number of different configurations or could be integrated into other parts of the handpiece 62 in some configurations.

As noted above, the drill bit 66 generally extends along the axis AX between the cutting tip portion 70 and the insertion portion 72, and is configured for releasable attachment to the handpiece 62 described herein and illustrated throughout the drawings via engagement between the interface 124 of the drill bit 66 and the bore 122 of the drive element 116 of the drive assembly 114. The drive element 116, in turn, cooperates with the output hub 96 of the gearset 86 of the actuator assembly 82 to facilitate rotating the drill bit 66 about the axis AX.

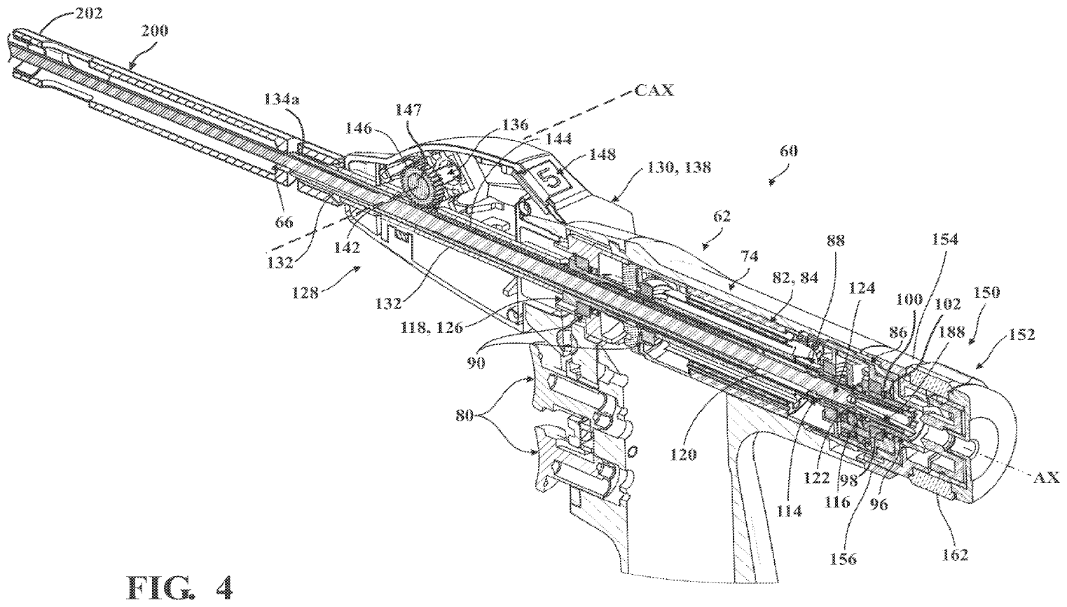

The illustrated configuration of the surgical drill system 60 further comprises the measurement module (alternatively referred to sometimes as a measurement head), generally indicated at 128, which may be configured to releasably attach to the handpiece 62 to provide the surgeon with measurement functionality during use. To this end, and as is best shown in FIGS. 4 and 5, the measurement module 128 may generally comprise a housing 130, a guide bushing 132, and a depth cannula 134 (i.e., a measurement probe, a depth measurement member, or a measurement cannula), which includes a distal end 134A adapted for placement against a workpiece, or tissue when used with a drill bit having a cutting portion smaller than an inner diameter of a lumen of the depth cannula 134. Suitable examples of a measurement module are described in International Patent Publication No. WO 2019/035096 A1, which is hereby incorporated by reference in its entirety. The housing 130 may be releasably attachable to the handpiece 62 and generally support the various components of the measurement module 128. The illustrated housing 130 may be formed as a pair of housing components 138 which interlock or otherwise attach together, and may be configured for disassembly to facilitate cleaning or servicing the measurement module 128. It should be appreciated that the measurement module 128 may be formed as an integral component of the handpiece 62, or may be in the form of a component that is affixed or otherwise secured to the handpiece 62 in a manner wherein the measurement module 128 is not removable from the handpiece 62 after use.

In the illustrated configuration, the housing components 138 and the guide bushing 132 comprise correspondingly-shaped features arranged to prevent relative axial and rotational movement therebetween, such as via notches formed in the guide bushing 132 which fit into webs or ribs formed in the housing components (not shown in detail). The guide bushing 132 may further comprises a window 142 as described in detail below.

The depth cannula 134 may be disposed within the guide bushing 132 and is supported for translational movement along the axis AX relative to the handpiece 62. An elongated recessed slot 143 (partially depicted in FIG. 2) may be formed transversely into the depth cannula 134 and extends longitudinally. While not specifically illustrated herein, the elongated recessed slot 143 may be shaped and arranged to receive a travel stop element which, in turn, is supported by the housing 130 and likewise extends through an aperture formed transversely through the side of the guide bushing 132. This arrangement may serve to limit how far the depth cannula 134 may be axially extended or retracted relative to the guide bushing 132 and housing 130, and may also prevent the depth cannula 134 from rotating about the axis AX. However, it will be appreciated that the measurement module 128 could be configured to limit or prevent movement of the depth cannula 134 in other ways without departing from the scope of the present disclosure.

As illustrated, the depth cannula 134 further comprises rack teeth 144 which are disposed in meshed engagement with a gear 146 of a transducer assembly 136. In some configurations, the transducer assembly 136 may be considered a displacement sensor. As shown in FIG. 5, the window 142 of the guide bushing 132 is arranged adjacent to the transducer assembly 136 to facilitate the meshed engagement between the rack teeth 144 and the gear 146. The gear 146 includes a shaft portion 147 extending along a common gear axis CAX. The gear 146 itself is rotatable 360 degrees about the common gear axis CAX as the probe 134 moves along the axis AX relative to the housing 130.

The transducer assembly 136 is responsive to rotation of the gear 146 resulting from axial movement of the measurement probe 134 in order to generate electrical signals (i.e., a transducer signal) representing changes in the position of the measurement probe 134 relative to the housing 130 along the axis AX, which correspond to the relative positioning of the distal end 134A of the depth cannula 134 relative to the housing 130 when the surgical drill 61 is placed against the workpiece. Thus, it will be appreciated that the transducer assembly 136 is able to provide the surgical handpiece assembly 62 with enhanced functionality. By way of example, in some configurations, the transducer assembly 136 may be disposed in communication with the controller 78, which may be configured to interrupt or adjust how the motor 84 is driven based on movement of the measurement probe 134, such as to slow rotation of the drill bit 66 at a specific drilling depth into the workpiece. The transducer assembly 136 may also be disposed in communication with an output device 148, such as a display screen, one or more light-emitting diodes (LEDs), and the like, to provide the surgeon with information relating to movement of the measurement probe 134, such as to display a real-time drilling depth, a recorded historical maximum drilling depth, and the like. Other configurations are contemplated. The output device 148 may be part of the measurement module that is removable. Further, while the transducer assembly 136 and the depth cannula 134 illustrated in FIG. 4 collectively comprise a rack and pinion design with the rack teeth 144 of the depth cannula 134 and the gear 146 of the transducer assembly 136, it is contemplated that the transducer assembly 136 may comprise one or more sensors such as a potentiometer, an optical sensor, and a linear variable displacement transformer to generate transducer signals responsive to displacement of the depth cannula 134 relative to the housing 130.

In many configurations, drill bits of varying sizes may be used to penetrate tissue for different procedures, e.g., a surgical procedure may require larger screws than screws for another surgical procedure. In such surgical procedures it may be necessary to use drill bits having different outer diameters of the cutting portion 70 of the drill bit 66 to establish larger holes to accommodate the larger screws. In such configurations requiring large cutting portions 70 of drill bits 66, the cannulated arrangement of the drive cannula 116, the depth cannula 134, the guide bushing 132, and the drill bit 66 may complicate the operation of depth measurement with drill bits 66 having large cutting portions 70. In some configurations, the outer diameter of the cutting portion 70 of the drill bit 66 may be larger than the inner diameter of the depth cannula 134. Employing a larger depth cannula 134 with an inner diameter sized to accommodate a larger cutting portion 70 may resolve dimensional concerns between the drill bit 66 and the depth cannula 134, however, the larger depth cannula 134 may create new sizing issues relating to the depth cannula 134 being received in the guide bushing 132 and the drive cannula 116. In other configurations, the outer diameter of the cutting portion 70 of the drill bit 66 may be larger than the inner diameter of the drive cannula 116. To address these concerns, a depth measurement extension 200 may be coupled to a distal portion of the depth cannula 134 to accommodate drill bits 66 having larger cutting portions 70 without compromising the cannulated arrangement of the surgical handpiece system 60.

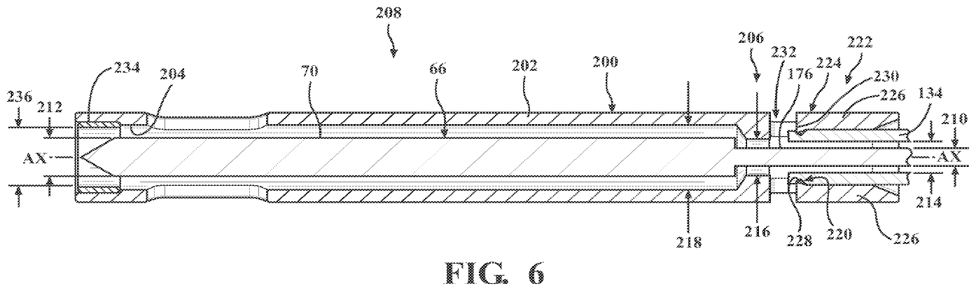

As shown in FIGS. 6-8B, the depth measurement extension 200 comprises an elongated body 202 having an inner surface 204 defining a bore. The bore extends between proximal and distal ends of the elongated body 202 and receives the drill bit 66. The bore is in communication with the lumen of the depth cannula 134 when the depth cannula 134 is coupled to the depth measurement extension 200. The elongated body 202 may comprise a monolithic structure such that the elongated body 202 comprise a single unitary construction. In other configurations, the elongated body 202 may comprise a plurality of components secured together. The elongated body 202 has a proximal portion 206 sized to accommodate passage of the shank portion 176 of the drill bit 66 through the bore. The proximal portion 206 may be sized to prevent passage of the cutting portion 70 of the drill bit 66 through the bore. Said differently, the proximal portion 206 of the elongated body 202 may be sized smaller than the cutting portion 70 of the drill bit 66 so that the cutting portion 70 cannot enter the distal end of the elongated body 202 and exit the proximal end of the elongated body 202. The elongated body 202 also includes a distal portion 208 that is sized to accommodate passage of the shank portion 176 and the cutting portion 70 of the drill bit 66 through the bore.

One advantage of having the proximal portion 206 sized to prevent passage of the cutting portion 70 of the drill bit 66 through the bore is to prevent the depth measurement extension 200 from unintentionally sliding distally relative to and away from the end of the drill bit 66 if the depth cannula 134 and the depth measurement extension 200 become detached. In other words, the relative sizing may capture the depth measurement extension 200 between the cutting portion 70 of the drill bit 66 and the depth cannula 134 in the occurrence of inadvertent detachment of the depth measurement extension 200 from the depth cannula 134.

The shank portion 176 of the drill bit 66 has a first outer diameter 210. The cutting portion 70 of the drill bit 66 has a second outer diameter 212. In configurations of drill bits 66 having second outer diameters 212 larger than an inner diameter 214 of the depth cannula 134, the second outer diameter 212 is larger than the first outer diameter 210. The proximal portion 206 of the elongated body 202 may have a first internal diameter 216 greater than the first outer diameter 210 and less than the second outer diameter 212 such that the cutting portion 70 of the drill bit 66 is prevented from passing through the bore of the elongated body 202. The distal portion 208 of the elongated body 202 may have a second internal diameter 218 greater than the first and second outer diameters 210, 212 to accommodate axial movement of the depth measurement extension 200 along the cutting portion 70 of the drill bit 66.

The depth cannula 134 may comprise a first coupler 220 and a coupling portion 222 of the depth measurement extension 200 may comprise a second coupler 224. The coupling portion 222 may be disposed proximal of the proximal portion 206. The first coupler 220 is configured to engage the second coupler 224 to facilitate removable coupling between the depth cannula 134 and the depth measurement member 200.

In one configuration illustrated in FIGS. 6-8B, the second coupler 224 may comprise one or more deflectable projections 226 and the first coupler 220 may define one or more recesses 228. The one or more recesses 228 may be configured to receive a portion of the one or more projections 226 to facilitate removable coupling between the depth cannula 134 and the depth measurement extension 200. More specifically, the one or more recesses 228 may be configured to receive a tab 230 of the one or more projections 226 that extends toward the bore. The second coupler 224 may define a cut-out 232 partially surrounding at least one of the one or more projections 226. The cut-out 232 assists the projection 226 in being deflectable relative to the bore. It is contemplated that the first and second couplers 220, 224 could be reversed such that the depth cannula 134 comprises the projections 226 and the depth measurement extension 200 defines the recess 228. It is also contemplated that the depth cannula 134 and the depth measurement extension 200 may be attached together in other manners including coupling via fasteners, threaded coupling, use of an adhesive, or in another manner.

The depth measurement extension 200 may include a bushing 234 disposed at least partially in the bore of the distal portion 208 of the elongated body 202. The bushing 234 may prevent the cutting portion 70 of the drill bit 66 from contacting the elongated body 202. The bushing 234 has an inner diameter 236 that is larger than the second outer diameter 212 of the cutting portion 70 of the drill bit 66.

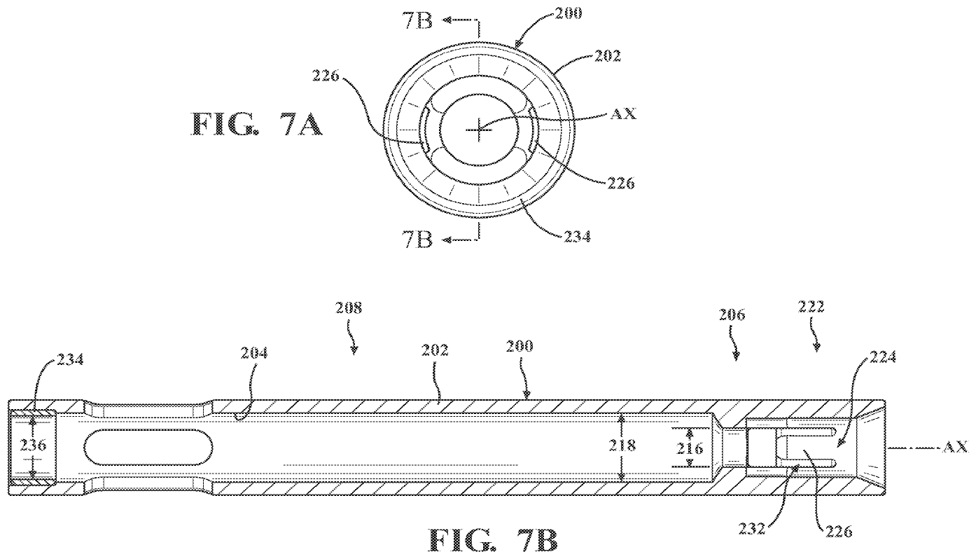

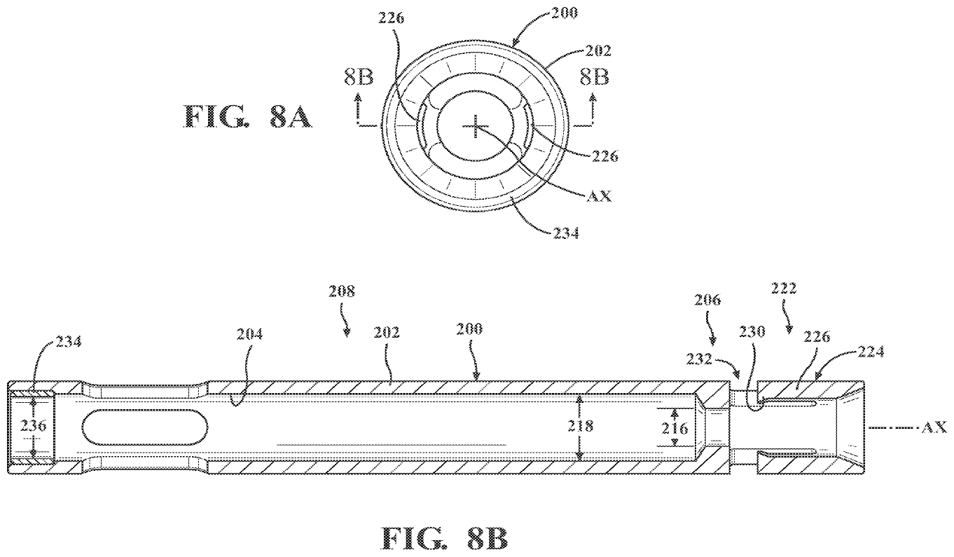

In the configuration illustrated in FIGS. 7A and 8A, the proximal portion 206 of the elongated body 202 has a circular inner surface. In other configurations, the proximal portion 206 of the elongated body 202 has a non-circular inner surface. In other configurations, the inner surface of the proximal portion 206 of the elongated body 202 may define an inscribed reference circle that is smaller than the second outer diameter 212 of the cutting portion 70 of the drill bit 66. Said differently, the diameter of the largest circle that can be disposed in the bore defined by the inner surface of the proximal portion 206 of the elongated body 202 has a diameter that is smaller than the second outer diameter 212 of the cutting portion 70 of the drill bit 66.

In another configuration, the proximal portion 206 of the elongated body 202 may have a first internal cross-sectional area for accommodating the shank portion 176 of the drill bit 66. The distal portion 208 of the elongated body 202 may have a second internal cross-sectional area for accommodating the cutting portion 70 of the drill bit 66. The second internal cross- sectional area may be greater than the first internal cross-sectional area. The coupling portion 222 of the elongated body 202 may have a third internal cross-sectional area for accommodating the depth cannula 134. The third internal cross-sectional area may be greater than the first internal cross-sectional area. The third internal cross-sectional area may be less than the second internal cross-sectional area. In configurations where the depth measurement extension 200 includes a bushing 234, the bushing 234 may have an internal diameter that is greater than the first internal cross-sectional area.

In an exemplary configuration, a caregiver can use the surgical handpiece system 60 for drill bits of different sizes such that attachment of the depth measurement extension 200 may be necessary for the drill bits 66 illustrated in FIG. 1 with cutting portions 70 larger than the inner diameter of the depth cannula 134. In such a configuration, operation of the depth cannula 134 for drill bits having cutting portions smaller than the inner diameter of the depth cannula 134 would function in a manner consistent with the surgical handpiece system described in previously mentioned International Patent Publication No. WO 2019/035096 A1. When a caregiver needs to use a drill bit 66 having a larger cutting portion 70, the user can load the larger drill bit 66 into the drive element 116 as described above and the insertion portion 72 and shank portion 176 may be identical to the insertion portion and shank portion of the smaller drill bit. In this way, coupling of the larger drill bit 66 to the surgical handpiece assembly 62 and operation of the surgical handpiece assembly 62 to rotate the larger drill bit 66 would be the same as coupling the smaller drill bit to the surgical handpiece assembly and operation of the surgical handpiece assembly to rotate the smaller drill bit.

Several configurations have been discussed in the foregoing description. However, the configurations discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

It will be further appreciated that the terms “include,” “includes,” and “including” have the same meaning as the terms “comprise,” “comprises,” and “comprising.” Moreover, it will be appreciated that terms such as “first,” “second,” “third,” and the like are used herein to differentiate certain structural features and components for the non-limiting, illustrative purposes of clarity and consistency.

1. A surgical handpiece system comprising:
a drill bit having a shank portion and a cutting portion;
a surgical handpiece assembly comprising,
a handpiece housing,
a motor disposed within the handpiece housing and configured to generate a torque,
a drive element disposed within the handpiece housing and configured to be coupled to the drill bit, the drive element configured to receive the torque from the motor and rotate the drill bit in response to the torque when the drill bit is coupled to the drive element,
a depth measurement member being moveable relative to the handpiece housing along an axis that is parallel to an axis of the drill bit, and
a displacement sensor for generating displacement signals responsive to movement of the depth measurement member relative to the handpiece housing; and
a depth measurement extension configured to be coupled to the depth measurement member, the depth measurement extension comprising,
an elongated body having an inner surface defining a bore extending between proximal and distal ends for receiving the drill bit, the elongated body comprising,
a proximal portion sized to accommodate passage of the shank portion of the drill bit through the bore, and the proximal portion sized to prevent passage of the cutting portion of the drill bit through the bore, and
a distal portion sized to accommodate passage of the shank portion and the cutting portion of the drill bit through the bore.

2. The surgical handpiece system of claim 1, wherein the shank portion of the drill bit has a first outer diameter and the cutting portion of the drill bit has a second outer diameter greater than the first outer diameter, and wherein the proximal portion of the elongated body has a first internal diameter greater than the first outer diameter and less than the second outer diameter, and wherein the distal portion of the elongated body has a second internal diameter greater than the first and second outer diameters.

3. The surgical handpiece system of claim 1, wherein one of the depth measurement extension and the depth measurement member comprises a first coupler and the other of the depth measurement extension and the depth measurement member comprises a second coupler, wherein the first coupler is configured to engage the second coupler to facilitate removable coupling of the depth measurement extension and the depth measurement member.

4. The surgical handpiece system of claim 3, wherein one of the first and second couplers has one or more deflectable projections, and wherein the other of the first and second coupler defines one or more recesses, each recess configured to receive a portion of one of the one or more projections to facilitate removable coupling between the depth measurement extension and the depth measurement member.

5. The surgical handpiece system of claim 4, wherein one of the first and second couplers defines a cut-out partially surrounding at least one of the one or more projections.

6. The surgical handpiece system of claim 1, wherein the depth measurement member comprises a cannula defining a lumen, the lumen of the cannula being in communication with the bore of the depth measurement extension when the depth measurement extension is coupled to the depth measurement member.

7. The surgical handpiece system of claim 1, wherein the depth measurement extension comprises a bushing disposed at least partially in the bore of the distal portion of the elongated body.

8. The surgical handpiece system of claim 1, wherein the elongated body comprises a monolithic structure.

9. The surgical handpiece system of claim 1, wherein the proximal portion of the elongated body has non-circular inner surface.

10. The surgical handpiece system of claim 1, wherein the drive element comprises a drive cannula.

11. A surgical handpiece system comprising:
a first drill bit having a first shank portion and a first cutting portion, the first cutting portion having a first outer diameter;
a second drill bit having a second shank portion and a second cutting portion, the second cutting portion having a second outer diameter greater than the first outer diameter;
a surgical handpiece assembly comprising,
a handpiece housing,
a motor disposed within the handpiece housing and configured to generate a torque,
a drive element disposed within the handpiece housing and configured to be coupled separately to each of the first and second drill bits, the drive element configured to receive the torque from the motor and rotate one of the first and second drill bits in response to the torque when one of the first and second drill bits is coupled to the drive element,
a depth cannula being moveable relative to the handpiece housing, the depth cannula defining a lumen extending along a length of the depth cannula, and the depth cannula sized to separately receive at least the first and second shank portions of the first and second drill bits, wherein the depth cannula has a first inner diameter greater than the first outer diameter of the first cutting portion of the first drill bit, and wherein the first inner diameter is less than the second outer diameter of the second cutting portion of the second drill bit.
a displacement sensor for generating displacement signals responsive to movement of the depth cannula relative to the handpiece housing; and
a depth measurement extension configured to be coupled to the depth cannula, the depth measurement extension comprising an elongated body, the elongated body having a distal portion having a second inner diameter greater than the first and second outer diameters of the first and second cutting portions of the first and second drill bits.

12. The surgical handpiece system of claim 11, wherein the first and second shank portions of the first and second drill bits each have equal shank outer diameters.

13. The surgical handpiece system of claim 11, wherein the drive element comprises a drive cannula.

14. A depth measurement extension for coupling to a depth measurement member of a surgical drilling system that includes a drill bit having a shank portion and a cutting portion, the depth measurement extension comprising:
an elongated body having an inner surface defining a bore extending between proximal and distal ends for receiving the drill bit and the depth measurement member, the elongated body comprising,
a proximal portion having a first internal cross-sectional area for accommodating the shank portion of the drill bit,
a distal portion having a second internal cross-sectional area for accommodating the cutting portion of the drill bit, the second internal cross-sectional area being greater than the first internal cross-sectional area, and
a coupling portion disposed proximal of the proximal portion, the coupling portion having a third cross-sectional area for accommodating the depth measurement member, the third internal cross-sectional area being greater than the first internal cross-sectional area.

15. The depth measurement extension of claim 14, wherein the coupling portion comprises one or more projections being deflectable into the bore for coupling the depth measurement extension to the depth measurement member.

16. The depth measurement extension of claim 15, wherein the coupling portion defines cut-outs partially surrounding the one or more projections.

17. The depth measurement extension of any one of claim 14, further comprising a bushing disposed at least partially in the bore of the distal portion of the elongated body.

18. The depth measurement extension of claim 17, wherein the bushing has an internal diameter that is greater than the first internal cross-sectional area.

19. The depth measurement extension of claim 14, wherein the elongated body comprises a monolithic structure.

20. The depth measurement extension of claim 14, wherein the first internal cross-sectional area of the proximal portion is non-circular.
